(19) United States
(12) Patent Application Publication
(10) Pub. No.: US 2015/0374274 A1
(43) Pub. Date: Dec. 31, 2015

(54) Oral Detection Apparatus

(71) Applicant: Ace Jovanovski, Round Rock, TX (US)

(72) Inventor: Ace Jovanovski, Round Rock, TX (US)

(21) Appl. No.: 14/319,685

(22) Filed: Jun. 30, 2014

(51) Int. Cl. A61B 5/00; A61B 5/145

(52) U.S. Cl. CPC A61B 5/4211; A61B 5/14507; A61B 5/0017; A61B 5/682

(57) ABSTRACT

An oral detection device is used for making oral detection of gastroesophageal reflux disease or other diseases or disorders. It includes a sensor, a sensor container, a base and a tooth retainer. The sensor is held in the sensor container and got its orientation maintained towards the esophageal opening. The tooth retainer works to connect the base where the sensor container and the sensor within are secured to, to teeth in either a maxillary style or a mandibular style. In this way, the oral detection device is able to detect gastroesophageal reflux or other targets in mouth in a removable and reusable way.

### FIELD OF THE INVENTION

The present invention relates generally to a detection apparatus that is able to perform detection in mouth. More specifically, it is an oral apparatus that can be attached onto teeth and thus to make detection in human mouth, as well as a method for utilizing the apparatus.

### BACKGROUND OF THE INVENTION

Many human diseases have their particular indicating observations or symptoms showing at their early stages in certain organs or locations of the body. Accordingly, an early detection of such physiological indicating signs can be greatly helpful to identify such diseases and subsequently treat the diseases in their early stages, which is able to provide precious health benefits to patients as well as save huge economic and social costs associated with the diseases. In this regard, a convenient and well functional device to facilitate such detection is always in need. On the other hand, since various diseases show their indicating signs in different organs or different locations of human body, a wide variety of detection devices is necessary to work at different locations of the body in order to carry out the various detections.

Moreover, since a certain detection device need to perform the detection at a particular location of the human body for a period of time, in addition to its detection functionality, such device's convenience and comfortability also play significant roles for the device's success and popularity among its targeted users. In this regard, a detection device that is able to provide notably improved convenience and/or comfortability would be able to meet patient's needs and provide new or improved health benefits.

As mentioned previously, many human diseases and disorders are associated with respective physiological signs in certain human body locations. Among them, gastroesophageal reflux disease (GERD) is a good example that has shown clear physiological signs. GERD is a chronic symptom of mucosal damage caused by stomach acid coming up from the stomach into the esophagus. It is already known that GERD is usually caused by changes in the barrier between the stomach and the esophagus, including abnormal relaxation of the lower esophageal sphincter, which normally holds the top of the stomach closed, impaired expulsion of gastric reflux from the esophagus, or a hiatal hernia.

As for the diagnosis of GERD, it should be noted that other causes of chest pain such as heart disease should be ruled out before making the final diagnosis. In addition, other investigations may include esophagogastroduodenoscopy (EGD). Barium swallow X-rays should not be used for diagnosis. Esophageal manometry is not recommended for use in its diagnosis, either. As a result, the current gold standard for diagnosis of GERD is esophageal reflux pH monitoring. It is the most objective test so far to diagnose the reflux disease; and it also allows monitoring GERD patients in their response to medical or surgical treatment subsequent to the diagnosis.

In regard to the gastroesophageal reflux disease (GERD), it has been defined as an involuntary muscle relaxing of the upper esophageal sphincter, which allows refluxed acid to move upward through the esophagus and into the oral cavity. In adults, GERD is a highly prevalent disease, with rates up to 56%. In addition, most of GERD is silent, or without major symptoms, which result in difficulties in its diagnosis. On the other hand, in many cases, dental professionals have the direct insight into the earliest evidence of GERD. For example, dental erosion is a progressive loss of hard dental tissues caused by chemical process. There are a few oral signs of GERD including hypersensitivity of teeth, change in color of teeth, and loss of enamel or dentin leading to a matte finish. Moreover, there are also some other oral symptoms that are associated with GERD, such as xerostomia, burning mouth syndrome, halitosis, and mucositis. GERD induced tooth wear results in rapid exposure of the dentin and pulpal tissues, and leading to shortened clinical crowns. Clinical rehabilitation of residual damaged dentition can often be technically challenging and time intensive. In light of the foregoing, an early diagnosis of the diseases such as GERD is the key to maintain healthy tooth structure, resulting in less invasive treatment modalities.

Furthermore, dentists are well known as one of the first health care professionals to diagnose a systemic disease through observation of its oral manifestations. As mentioned previously, GERD is a disease that may be evidenced by dental erosion. Untreated GERD may result in more serious conditions such as Barrett's esophagitis, and it may also increase a patient's risk for cancer. Accordingly, it is important that dentists help to recognize GERD whenever it is possible, such that appropriate treatment modalities can be timely instituted. As a result, the collaborative medical and dental management of patients with GERD is strongly advocated.

Until recently, the major approach for diagnosing GERD was pH testing via ambulatory catheter pH metry monitoring. However, such conventional catheter pH metry may cause undue burdens on the patients, at least partially due to the discomfort and embarrassment of the transnasal pH probe placement. Many patients have reported modification of daily activities and/or diet with this commonly used approach.

Accordingly, certain medical devices that are more convenient have been recently developed for such pH testing. For instance, the Bravo™ pH Monitoring System (Medtronic, Minneapolis, Minn.) is the first catheter-free approach for measuring esophageal pH levels in patients who have or are suspected of having GERD. With the Bravo system, patients can maintain their usual diet and activities while having their pH levels tested. Also, there are no cumbersome tubes or wires for the patient to deal with. The Bravo™ system involves a miniature pH capsule, about the size of a gelcap, attached to a patient's esophagus. The capsule wirelessly transmits pH testing information to a pager-sized receiver worn by the patient. After the test is completed, data from the receiver would be uploaded to pH analysis software using infrared technology.

However, although the Bravo™ capsule, a major part of the Bravo™ pH Monitoring System, is a device that has been designed to overcome the above mentioned disadvantages and inconveniences associated with the traditional pH monitoring approaches, it has its own technical problems and side effects. One of the primary technical problems of this test is related to failures in transmission, or early detachment of the Bravo™ capsule. In addition, there are many case reports that describe a list of other unusual problems, such as severe chest pain that necessitate immediate removal. Moreover, the screening and diagnosis are usually performed by the gastroenterologist, which limits the total number of patients that can benefit from early treatment.

In light of the foregoing, one objective of the present invention is to provide a detection apparatus as well as a method of using the same with the purpose of facilitating such pH testing for GERD. The detection via this apparatus is easy, convenient and secure, thus essentially solves the issues such as transmission failure and early capsule or sensor detachment. In addition, such testing could be easily performed by dentists, who have wider patient basis than the gastroenterologist. As a result, the present invention would have the potential of offering nearly over 200,000 dentists in North American the potential to screen, diagnose, or refer patients that suffer from GERD.

### BRIEF DESCRIPTION OF THE DRAWINGS

FIG. 1 is a bottom view (from the back) of the present invention in an upper (maxillary) style.

FIG. 2 is left view (left panel) and right view (right panel) of the present invention in an upper (maxillary) style.

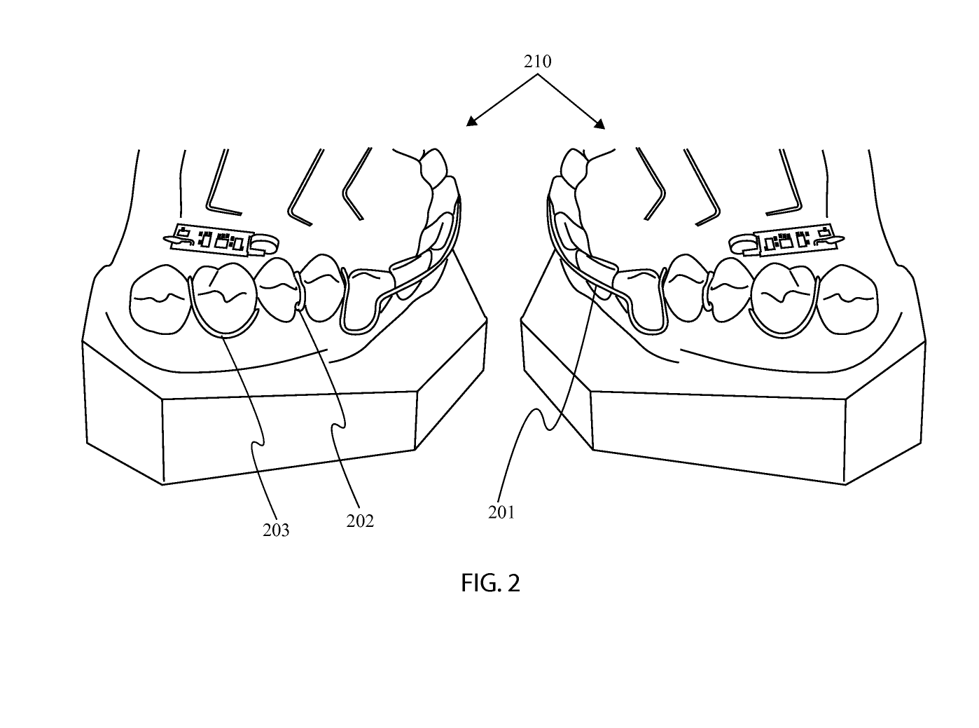

FIG. 3 is a bottom view (from the front) of the present invention in an upper (maxillary) style.

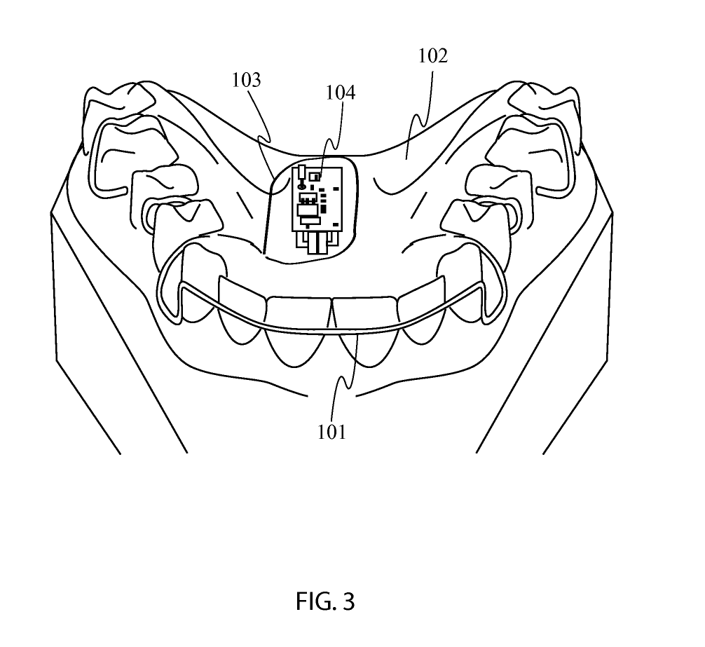

FIG. 4 is a perspective view of the present invention in an upper (maxillary) style.

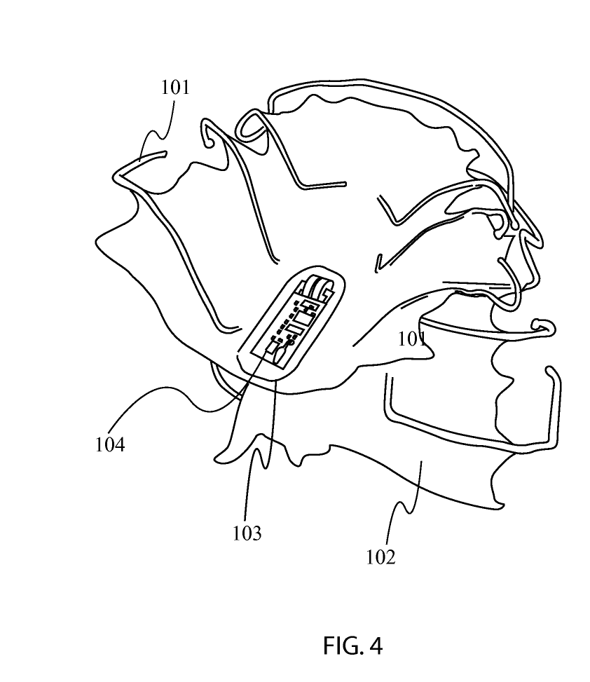

FIG. 5 is a top view (from the back) of the present invention in a lower (mandibular) style.

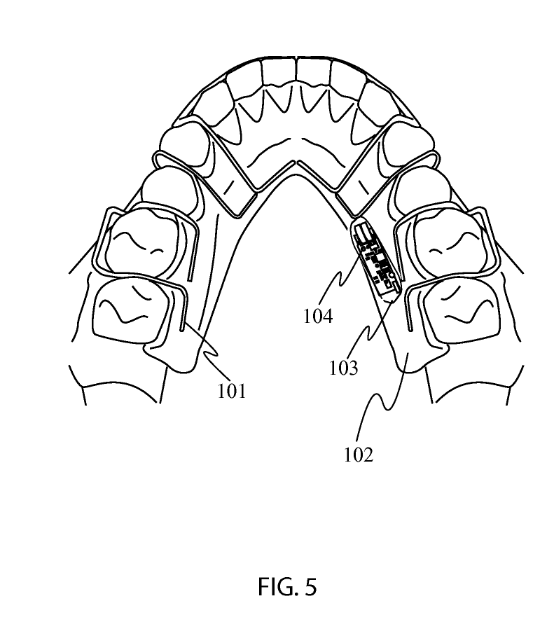

FIG. 6 is right view (left panel) and left view (right panel) of the present invention in a lower (mandibular) style.

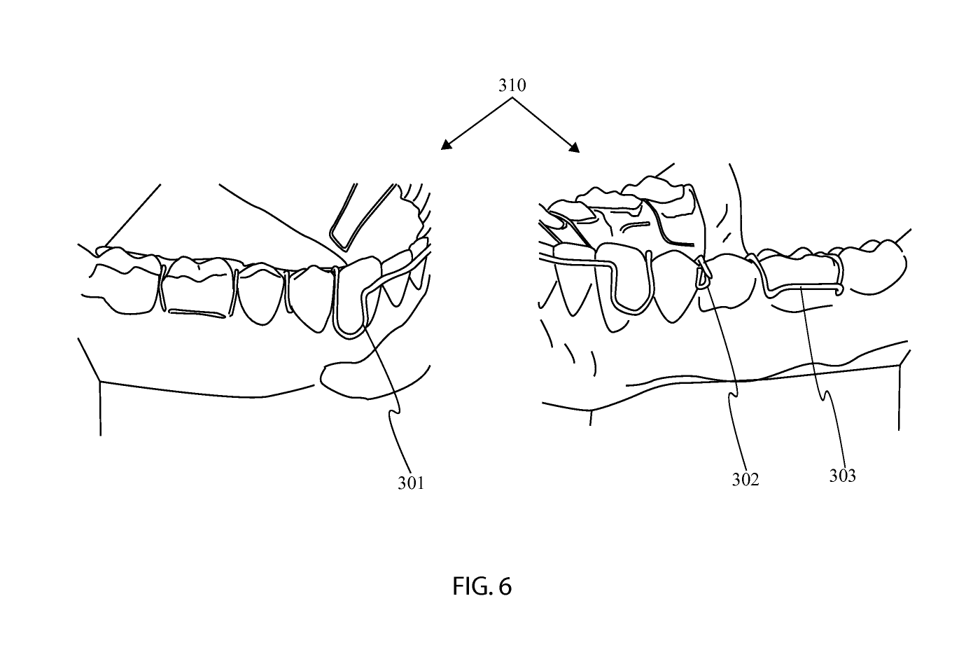

FIG. 7 is a top view (from the front) of the present invention in a lower (mandibular) style.

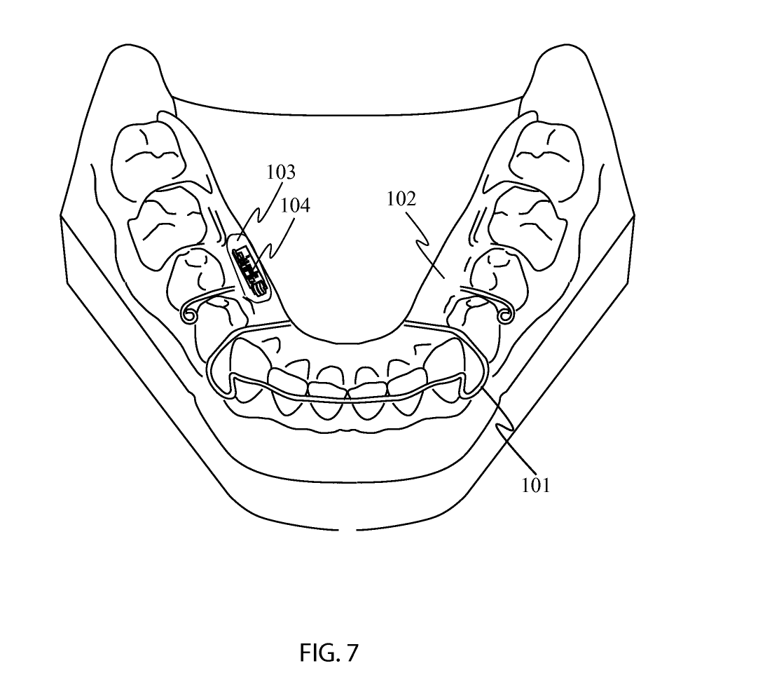

### DETAIL DESCRIPTIONS OF THE INVENTION

All illustrations of the drawings are for the purpose of describing selected versions of the present invention and are not intended to limit the scope of the present invention.

The present invention is an oral detection device which has been adapted to perform certain testing in human mouth, such as the pH value testing that is the key for early diagnosis for diseases and disorders including gastroesophageal reflux disease (GERD). In addition, in the present invention, it has also provided a method of efficiently using this oral device. It is noted that although the oral device disclosed in the present invention has been adapted for the oral pH value testing targeting the diseases of GERD, the device introduced in the present invention is also capable of facilitating the testing or detection of many other diseases or disorders, as well as other testing or detections that are not obviously associated with a particular diseases. In this regard, the present invention may have much broader applications beyond the detection of GERD.

In comparison to the currently available devices for detecting the reflux of GERD, the oral device of the present invention is easy to use. Accordingly, the present invention could be used as an alternative to the traditional pH testing method, which involves placing a catheter through the nose and down the esophagus. The transnasal catheter is uncomfortable and conspicuous, leading most patients to modify their daily activities and/or diet, or in some cases, to avoid the test altogether. The present invention does not need any extra intrusive equipment such as catheter to achieve the purpose of reflux testing, which is a significant advantage and thus provides welcomed beneficial effects to the patient.

Furthermore, the data collected with the oral device of the present invention are generally more representative of the patient's day to day activities than traditional pH monitoring methods because patients are able to maintain a normal diet and activity level during testing. In addition, while traditional pH tests are usually conducted for 24 hours, the oral device of the present invention allows physicians to monitor the reflux in a much longer time, such as a few days. It is noted that due to their particular designs, other catheter free systems such as Bravo are usually only perform a detection of 24 to 48 hours, let alone the detachment issue associated with such devices, which could make their detection time periods significantly reduced or even unpredictable.

On the other hand, the oral device of the present invention is much more secure and reliable. In other devices for the same purpose, the pH capsule about the size of a gelcap is attached to the wall of the esophagus and transmits data to the receiver. This apparently would lead to frequent occurrence of early detachment, since only the attachment at the very beginning could be performed and thus controlled by a surgeon; then after that, when and how the device finally falls (sloughs) off from the patient's esophagus wall is just a spontaneous and natural and spontaneous process that no one can control. Nevertheless, in the present invention, the entire process of when and how the device is installed and how and when the oral device is removed from the human body (mouth) are completely controlled by human, including the professionals and the patients themselves. In addition, the attachment of the oral device of the present invention to the human body is very secure, which essentially rule out the failure of early detachment.

What's more, the oral device of the preset invention can be more easily introduced into the human body (mouth). In regard to certain other recently developed devices that do not need a catheter, they still need certain specific costly surgical procedure to place the testing sensor onto the human esophagus. In many cases, the first step for using such devices involves a gastroenterologist, endoscopist or surgeon places the respective detection devices. The procedure can be performed in a clinic, hospital endoscopy lab or ambulatory surgery center.

While with using the oral device of the present invention, no any surgical procedure is needed, a dentist can easily help to install the device in a patient's mouth. As a result, it has saved a significant amount of time and costs, let alone a much relaxed medical experience, which is priceless.

The oral detection apparatus of the present invention, the Bravo ACE appliance, is a unique and efficient oral detection device that allows for screening of GERD or other disorders or diseases by a dental professional. The Bravo ACE appliance is a custom made maxillary, or mandibular oral apparatus with a sensor, such as the Bravo™ capsule embedded. This removable apparatus can be made to fit around the existing teeth, without any surgical intervention. As a result, the oral detection device of the present invention needs no ambulatory visit. In addition, since both installation and removal of the oral apparatus can be accurately control by a professional or patient, the oral detection apparatus of the present invention is able to be reused in follow-up studies. Since it is not a hospital or surgical based approach and it can be reused for many times, its costs have been significantly reduced.

One most beneficial feature of the present invention is that it is an oral detection device, which suggests that it could be easily used in human mouth, rather than in other organ or locations such as human esophagus, which are usually not that easy and convenient.

In general, the present invention is an oral detection apparatus with multiple components to carry respective functions, and working together, they form a unique apparatus that bring in desirable convenience and comfortability to reflux detection. As a detection apparatus, the present invention could be used for testing the acidity in a flux, which is the key in GERD diagnosis; though apparently, it can be used in any applications that need similar detections.

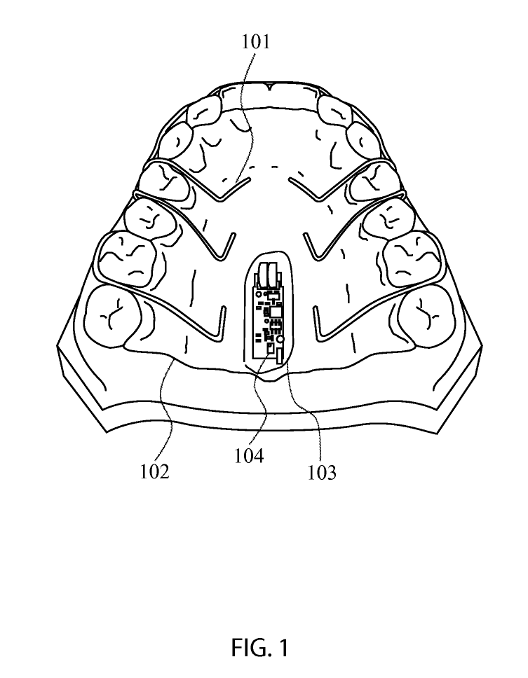

In reference to FIG. 1, the oral detection apparatus mainly comprises four essential components, namely, a detection sensor 104, a sensor container 103, a base 102 and a tooth retainer 101. In the present invention, the four have been integrated together to carry out the function of performing detection or testing in a human mouth.

The sensor 104 in the present invention could be a pH sensor. However, it could be other types of sensor which are used in other testing or detections. On the other hand, the sensor should be suitable for an oral detection. First, the sensor overall as well as the materials used to make it should be nontoxic to human being. Second, its materials should not be dissolved in the saliva. Third, the sensor should function properly in the environment of human mouth, which is dominated by the secreted saliva. It is noted that the human mouth environment is a dynamically changing one. For example, the pH value in a normal person's mouth could be quickly changed from as low as 6.4 or even lower to 7.8 or even higher. Accordingly, the sensor used in a human house should tolerate such changes and still functions properly. Third, the sensor should be compact and light weight, which allows the sensor to be easily introduced into the targeted locations in human body, such as mouth, as well as allows it to stay at such location securely during the time period of testing and make little uncomfortable issue to the patient who is using it.

The sensor container 103 is a capsule of the sensor. It also works together with other components to fix the sensor into mouth. Since it is going to be used in human mouth, the size of the capsule should be just big enough to accommodate the sensor, but not too big that may lead to being uncomfortable in human mouth. In addition, inside the sensor container, there is a receiving mechanism to securely hold the sensor. So, the sensor will not fall out of the capsule.

Moreover, the container has an opening to the oral environment, which is big enough to allow the sensor in it to get access to the mouth environment, especially to the reflux that the sensor is going to detect. In this way, the data obtained via the sensor within the capsule would be more reliable and consistent.

As mentioned previously, the sensor is held in this container securely. With the opening on the capsule or container, the sensor is able to get access to the reflux or other substances in human mouth. Furthermore, the orientation of the sensor in the capsule is also very important. Ideally, the sensor's transmitter antenna should be made sure to face as distal as possible. That is to say, the sensor transmitter antenna should be towards the esophageal opening (towards uvula).

The container 103 could be made of any material that is suitable for its functionality in the mouth. It should be nontoxic and free of undesirable taste or smell. In addition, it also should be soft and light weighted, which helps to minimize the foreign body sensation which frequently occurs following a foreign body being introduced into a specific location of human body. This is particularly true since human mouth is pretty sensitive. On the other hand, the container along with the sensor in it will stay in human mouth for a quite long time in order to obtain reliable data that faithfully represent the true reflux pH value of a patient. Moreover, when the reflux would come out is completely unpredictable. As a result, the duration that the device needs to stay in a patent's mouth may be pretty long. All of these factors demand the material used to make the sensor container to be safe, stable as well as comfortable.

The base 102 is one major component of the oral detection device. It is in contact with the roof of the human mouth, namely the palate, so as to stabilize the capsule that is connected to it, as well as the sensor contained in the capsule. On its surface, the base has certain microstructures that are corresponding to the shapes of human palate.

Concerning the material that can be used to make the base, any suitable material could be used in the present invention to make the base. Such material should be non-toxic, insoluble and stable. For example, acrylic resin would be a suitable material for making base of the oral detection apparatus. Acrylic resins are a group of related thermoplastic or thermosetting plastic substances derived from acrylic acid, methacrylic acid or other related compounds. One family of acrylic resin is polymethyl methacrylate which is used to make hard plastics with various light transmitting properties. Acrylic resin has a few desirable physical and chemical properties. It is pretty stable and strong. It is water resistant and cracking resistant. On the other hand, it has an excellent light transmitting performance. The foregoing properties make acrylic resin one of the ideal materials to make the base of present invention.

Furthermore, since the oral detection apparatus of the present invention is going to stay in human mouth for a relatively long period of time, in order to make its users willing to have it in the mouth, the base of the oral detection apparatus has been designed to have the color and feel of human palate. So, the color of the base is usually flesh pink. But other suitable color may also be selected. In this way, the awkwardness associate with wearing a detection apparatus in mouth has been significantly reduced, thus the user would be willing to use the oral detection apparatus of the present invention.

The last primary component of the present invention is a tooth retainer 101, which works together with the base to securely maintain the detection sensor and capsule in mouth for a considerably long period of time, in order to detect the reflux or perform other detections. As for the tooth retainer, according to specific actual situation and preference of individual patients, the tooth retainer may be held in an upper position maxillary style 210 or a lower position mandibular style 310.

The tooth retainer of the present invention may be made of metal or other suitable materials. It is usually made in a metal wire form. The metal wire could be wrought wire, stainless steel wire or titanium alloys and the like. Such metal wire is usually with a cross section in a shape of round or half round. The gauge of the metal wire is conventionally represented by a gauge number according to the American wire gauge (AWG), also known as the Brown & Sharpe wire gauge, which is a standardized wire gauge system used since 1857 predominantly in North America for solid metal wire. In the present invention, the metal wire of tooth retainer could be from gauge 12 to gauge 20, wherein the gauge 12 is of a diameter of 0.0808 inch, while the gauge 20 is of a diameter of 0.0320 inch.

In regard to the upper tooth retainer, it is also called as maxillary style tooth retainer. More specifically, the metal wire of the maxillary tooth retainer may comprise three small wire components, namely, the labial bow, the ball clasps and the C-claps. It is noted that the foregoing three small wire components are not necessarily connecting to each other. Rather, they may be individual wire components and each of them is securely fixed into the base. In this way, albeit they are not interconnected to each other, due to their respective connections with the base, they, along with the base, are still form one integral body of wires and base.

Labial bow 201 is a structure of metal wire which provides facial retention and resistance side to side. On one side, the labial bow with its two ends is securely fixed into the base; the other side of the labial bow is placed from canine to canine on the upper arch. The labial bow is usually one long piece of metal wire structure that spans from left side to right side. The ball clasps 202 are two pieces of small metal wire components. They are usually identical to each other and located at left and right side, respectively. Similarly, at one end, each of the ball clasps is securely fixed into the base; while at the other end, the two ball clasps are located in between the first premolar and the second premolar on the left side and the right side of the upper arch, respectively. The third metal wire component is the C-clasp 203. The C-clasp receives its name from its letter C-like shape. The C-clasps are also two pieces of small metal wire components. They are usually are two similar pieces located at left and right side, respectively. Similarly, at one end, each of the ball clasps is securely fixed into the base; while at the other end, the two

C-clasps are clasping the exterior portion of the first molar on the left or right side of the upper arch, respectively. Furthermore, placement (location) of the clasps is dependent on the height of contour of the actual tooth, while their rigidity is dependent on the path of insertion and or removal. It is also noted that albeit the two ball clasps are similar to each other and the two C-clasps are similar to each other as well, they are actually not identical. This is because they are all custom-made and barely a user's left side and right side teeth are exactly the same.

In regard to the lower tooth retainer, it is also called as mandibular tooth retainer. More specifically, the metal wire of the mandibular tooth retainer may comprise three small wire components, namely, the labial bow 301, the triangular clasps 302 and the Adams claps 303. It is noted that the foregoing three small wire components are not necessarily connecting to each other. Rather, they may be individual wire components and each of them is securely fixed into the base. In this way, albeit they are not interconnected to each other, due to their connections to the base, they, along with the base, are still form one integral body of wires and base.

The Labial bow 301 in the mandibular tooth container is similar to its counterpart in the maxillary tooth retainer, except for that at one end the wire of labial bow is placed from canine to canine on the lower arch, rather than the upper arch. The triangular clasps 302 are two pieces of small metal wire components. They are usually similar to each other and located at left and right side of the lower arch, respectively. At one end, each of the ball clasps is securely fixed into the base; while on the other side, the two triangular clasps are located in between the first premolar and the second premolar on the left side and the right side of the lower arch, respectively. Moreover, at its end between the premolar teeth, the horizontally lying triangle continues into a vertical part, then into the transversal tag and finally ends in the retention portion. In order to protect the cheek, the open part of the triangle should face distally. The third metal wire component is the Adams clasps 303. The Adams clasps are also two pieces of small metal wire components. They are usually two similar pieces located at left and right side of the lower arch, respectively. Similarly, at one end, each of the ball clasps is securely fixed into the base; while on the other side, the two Adams clasps are clasping the exterior portion of one of the molars on the left or right side of the lower arch, respectively. The Adams clasps have the function of anchorage, usually on molars. Each of them comprises a horizontal part and two U shaped loops that merge from the transversal clasp parts to the retention. The clinical crown determines the height of the loops. These loops should have such height that the horizontal part of the clasp runs 1 to 2 mm below the cusps.

In the oral detection device of the present invention, it has adopted a Hawley style wrap and design. That is to say, when clasps interfere with occlusion, a single wire goes across the span of the arch. The present invention is a removable oral detection device. It can be of full coverage, or of horseshoe style coverage. In addition, it can be used to conduct oral detection for users with a wide variety of tooth conditions. A user's teeth may be in one of the conditions as follows, full complement of teeth (dentate), several missing teeth (partially edentulous), all missing teeth (fully edentulous), or children or adolescent (primary dentition).

Furthermore, the present application also includes a method of using the oral detection device. In this process, the first step would be providing a sensor and a capsule, wherein the sensor is maintained in the capsule, next providing a base and a tooth container, wherein the tooth container has been securely fixed into the base, and the capsule has been securely fixed onto the base. In this way, the oral detection device of the present invention has form an integral body with four components, namely sensor, capsule, base and tooth retainer.

Subsequently, the oral detection device is placed on either the upper arch (in such a case, the oral detection device takes a maxillary style), or lower arch (in such a case, the oral detection device takes a mandibular style) of a user's mouth. As for the maxillary form, its tooth retainer comprises labial bow, ball clasps and C-clasps; whereas for the mandibular form, its tooth retainer comprises labial bow, triangular clasps and Adams clasps. Further, it is a key to make the sensor face towards the esophageal opening (towards uvula). Such that the sensor may catch the information from the reflux before it has been diluted by the saliva in the user's mouth. During the detection, the information of reflux is send wirelessly to a receiving device via infrared or other suitable approaches.

After a predetermined time period of detection, the oral detection device will be removed from the user's mouth, and saved for future reuse. The data stored in the receiving device will be examined by a professional for diagnosis or other purposes. In a case more reflux detection is deemed as necessary, the same oral detection device will be used again to perform the reflux detection in the user's mouth, and so on. As long as the sensor still functions properly, the oral detection apparatus of the present invention could be reused for indefinite times.

Moreover, as noted previously, the application of the oral detection device on the present invention is not limited to just perform the pH value detection for GERD. In addition, the same oral detection apparatus of the present invention can be used for many other examinations and detections that need to be performed in a patient's mouse for a considerably long period of time. In this context, the oral detection apparatus can be easily refitted to the respective detection via a number of different approaches. For example, if the sensor is capable of performing the new detection, then the only necessary modification is to reprogram the sensor to make it suitable for the new detection. In another case, if the sensor is not able to make the new detection, yet the capsule is suitable to the sensor for new detection, then the only modification is to replace the sensor with a different one. In such a case, at least three main components of the present invention, the capsule, the base and the tooth retainer will be reused in the new detection. In a scenario when the capsule does not match the new sensor, the capsule and sensor would be replaced, yet the previous base and tooth retainer will remain in the new oral detection apparatus.

Accordingly, the oral detection apparatus of the present invention would provide a few significant benefits to a user. First, it has been held in a user's mouth which is much more accessible and convenient than the wall of esophagus. As a result, in most cases, a user is able to do the installation and removal of the oral detection device, without requiring a professional to do it through a surgery. Most people are capable to put something in mouth on her or her own teeth; yet probably very few people are able to place some device on the wall of his or her own esophagus, let alone do it safely, securely and accurately at a specific location. Therefore, the oral detection device of the present invention can save its user a lot of money and time. And at the same time, it is more easy, convenient and comfortable. Second, many parts of the oral detection apparatus, including the base and tooth retainer, and may also including the capsule or even the sensor, can be reused by the user for many times, for the same detection or different detections. In this way, the oral detection device of the present invention is able to further save its user significant time and money in the long run.

Although the invention has been explained in relation to its preferred embodiments, it is to be understood that many other possible modifications and variations can be made without departing from the spirit and scope of the invention as herein described.

What is claimed is:

1. A removable and reusable oral detection device, comprising
a sensor;
a sensor container;
a base;
a tooth retainer;
said sensor being located in said sensor container;
said sensor container being bonded to said base; and
said tooth retainer being securely fixed on teeth in a mouth.

2. The removable and reusable oral detection device of claim 1, comprising
said sensor functioning in a human mouth environment;
said sensor being resistant to saliva; and
said sensor being nontoxic to human.

3. The removable and reusable oral detection device of claim 1, comprising
said sensor detecting gastroesophageal reflux; and
said sensor being a pH value detector.

4. The removable and reusable oral detection device of claim 1, comprising
said sensor container securely holding said sensor and maintain said sensor's orientation;
said sensor container being made of a nontoxic and saliva resistant material;
said sensor container comprising an opening; and
said opening allowing said sensor to get access to a target to be detected.

5. The removable and reusable oral detection device of claim 4, comprising
said sensor container being made of acrylic resin; and
said target is gastroesophageal reflux.

6. The removable and reusable oral detection device of claim 1, comprising
said base being made of a nontoxic and saliva resistant material; and
said base is stainable for a plurality of colors.

7. The removable and reusable oral detection device of claim 6, comprising
said based being made of acrylic resin; and
at least one of said plurality of colors being a color of human palate.

8. The removable and reusable oral detection device of claim 1, comprising
said tooth retainer being made of a nontoxic material comprising metal; and
said tooth retainer being in a shape of wire with a cross section of round or half round.

9. The removable and reusable oral detection device of claim 8, comprising
said metal being selected from the group consisting of wrought iron, stainless steel, titanium or titanium alloy; and
said cross section having a diameter within the range of AWG 12 to AWG 20, wherein AWG stands for American Wire Gauge.

10. The removable and reusable oral detection device of claim 1, comprising
said tooth retainer being in one of two styles;
said two styles comprising a maxillary style and a mandibular style;
said tooth retainer being attached to upper arch of teeth when in said maxillary style; and
said tooth retainer being attached to lower arch of teeth when in said mandibular style.

11. The removable and reusable oral detection device of claim 10, comprising
said tooth retainer in said maxillary style comprising a labial bow, two ball clasps and two C-claps;
said labial bow being a piece of metal wire;
said labial bow providing facial retention;
said labial bow comprising two ends securely embedded into said base;
said labial bow spanning from left canine tooth to right canine tooth of said upper arch of teeth;
said two ball clasps comprising a left ball clasp and a right ball clasp;
said left ball clasp comprising a first left ball clasp end and a second left ball clasp end;
said first left ball clasp end being securely embedded into said base;
said second left ball clasp end being located between first left premolar tooth and second left premolar tooth of said upper arch of teeth;
said right ball clasp comprising a first right ball clasp end and a second right ball clasp end;
said first right ball clasp end being securely embedded into said base;
said second right ball clasp end being located between first right premolar tooth and second right premolar tooth of said upper arch of teeth;
said two C-clasps comprising a left C-clasp and a right C-clasp;
said left C-clasp comprising a first left C-clasp end and a second left C-clasp end;
said first left C-clasp end being securely embedded into said base;
said second left C-clasp end clasping first left molar teeth of said upper arch of teeth;
said right C-clasp comprising a first right C-clasp end and a second right C-clasp end;
said first right C-clasp end being securely embedded into said base; and
said second right C-clasp end clasping first right molar teeth of said upper arch of teeth.

12. The removable and reusable oral detection device of claim 10, comprising
said tooth retainer in said mandibular style comprising a labial bow, two triangular clasps and two Adams claps;
said labial bow being a piece of metal wire;
said labial bow providing facial retention;
said labial bow comprising two ends securely embedded into said base;
said labial bow spanning from left canine tooth to right canine tooth of said lower arch of teeth;
said two triangular clasps comprising a left triangular clasp and a right triangular clasp;
said left triangular clasp comprising a first left triangular clasp end and a second left triangular clasp end;
said first left triangular clasp end being securely embedded into said base;
said second left triangular clasp end being located between first left premolar tooth and second left premolar tooth of said lower arch of teeth;
said right triangular clasp comprising a first right triangular clasp end and a second right triangular clasp end;
said first right triangular clasp end being securely embedded into said base;
said second right triangular clasp end being located between first right premolar tooth and second right premolar tooth of said lower arch of teeth;
said two Adams clasps comprising a left Adams clasp and a right Adams clasp;
said left Adams clasp comprising a first left Adams clasp end and a second left Adams clasp end;
said first left Adams clasp end being securely embedded into said base;
said second left Adams clasp end clasping first or second left molar tooth of said lower arch of teeth;
said right Adams clasp comprising a first right Adams clasp end and a second right Adams clasp end;
said first right Adams clasp end being securely embedded into said base; and
said second right Adams clasp end clasping first or second right molar teeth of said lower arch of teeth.

13. A method of using a removable and reusable oral detection device to perform a detection in mouth, comprising
providing a sensor;
providing a sensor container;
providing a base;
providing a tooth retainer;
securing said sensor into said sensor container;
fixing said sensor container to said base;
fixing said tooth retainer to said base;
determining a style to attach said oral detection device to teeth; and
attaching said tooth retainer to said teeth;
adjusting an orientation of transmitter antenna of said sensor to face esophageal opening;
starting said oral detection device;
receiving data from said oral detection device by a receiving device coupled to said oral detection device via a wireless connection;
saving said data in said receiving device;
performing detection for a predetermined time period;
removing said oral detection device from mouth; and
analyzing the saved data.

14. The method of using a removable and reusable oral detection device to perform a detection in mouth as claimed in claim 13, when said style to attach said oral detection device to teeth is determined to be a maxillary style, said step of attaching said tooth retainer to said teeth further comprising
providing a labial bow, two ball clasps and two C-claps to said tooth retainer, wherein said two ball clasps comprise a left ball clasp and a right ball clasp, said left ball clasp comprises a first left ball clasp end and a second left ball clasp end and said right ball clasp comprises a first right ball clasp end and a second right ball clasp end, said two C-clasps comprises a left C-clasp and a right C-clasp, said left C-clasp comprises a first left C-clasp end and a second left C-clasp end, and said right C-clasp comprises a first right C-clasp end and a second right C-clasp end;
spanning said labial bow from left canine tooth to right canine tooth of upper arch of teeth;
clasping said second left ball clasp end between first left premolar tooth and second left premolar tooth of said upper arch of teeth;
clasping said second right ball clasp end between first right premolar tooth and second right premolar tooth of said upper arch of teeth;
clasping said second left C-clasp end to first left molar teeth of said upper arch of teeth; and
clasping said second right C-clasp end to first right molar teeth of said upper arch of teeth.

15. The method of using a removable and reusable oral detection device to perform a detection in mouth as claimed in claim 13, when said style to attach said oral detection device to teeth is determined to be a mandibular style, said step of attaching said tooth retainer to said teeth further comprising
providing a labial bow, two triangular clasps and two Adams claps to said tooth retainer, wherein said two triangular clasps comprise a left triangular clasp and a right triangular clasp, said left triangular clasp comprises a first left triangular clasp end and a second left triangular clasp end and said right triangular clasp comprises a first right triangular clasp end and a second right triangular clasp end, said two Adams clasps comprises a left Adams clasp and a right Adams clasp, said left Adams clasp comprises a first left Adams clasp end and a second left Adams clasp end, and said right Adams clasp comprises a first right Adams clasp end and a second right Adams clasp end;
spanning said labial bow from left canine tooth to right canine tooth of lower arch of teeth;
clasping said second left triangular clasp end between first left premolar tooth and second left premolar tooth of said lower arch of teeth;
clasping said second right triangular clasp end between first right premolar tooth and second right premolar tooth of said lower arch of teeth;
clasping said second left Adams clasp end to first or second left molar teeth of said lower arch of teeth; and
clasping said second right Adams clasp end to first or second right molar teeth of said lower arch of teeth.

16. The method of using a removable and reusable oral detection device to perform a detection in mouth as claimed in claim 13,
said wireless connection being an infrared connection.

17. A removable and reusable oral detection device, comprising
a sensor;
a sensor container;
a base;
a tooth retainer;
said sensor being located in said sensor container;
said sensor container being bonded to said base;
said tooth retainer being securely fixed on teeth in a mouth;
said tooth retainer being in one of two styles;
said two styles comprising a maxillary style and a mandibular style;
said tooth retainer being attached to upper arch of teeth when in said maxillary style; and
said tooth retainer being attached to lower arch of teeth when in said mandibular style.

18. The removable and reusable oral detection device of claim 17, comprising
said sensor functioning in a human mouth environment;
said sensor being resistant to saliva;
said sensor being nontoxic to human;
said sensor detecting gastroesophageal reflux;
said sensor being a pH value detector;
said sensor container securely holding said sensor and maintain said sensor's orientation;
said sensor container being made of a nontoxic and saliva resistant material;
said sensor container comprising an opening;
said opening allowing said sensor to get access to a target to be detected;
said sensor container being made of acrylic resin;
said target is gastroesophageal reflux;
said base being made of a nontoxic and saliva resistant material;
said base is stainable for a plurality of colors;
said based being made of acrylic resin;
at least one of said plurality of colors being a color of human palate;
said tooth retainer being made of a nontoxic material comprising metal;
said tooth retainer being in a shape of wire with a cross section of round or half round;
said metal being selected from the group consisting of wrought iron, stainless steel, titanium or titanium alloy; and
said cross section having a diameter within the range of AWG 12 to AWG 20, wherein AWG stands for American Wire Gauge.

19. The removable and reusable oral detection device of claim 18, comprising
said tooth retainer in said maxillary style comprising a labial bow, two ball clasps and two C-claps;
said labial bow being a piece of metal wire;
said labial bow providing facial retention;
said labial bow comprising two ends securely embedded into said base;
said labial bow spanning from left canine tooth to right canine tooth of said upper arch of teeth;
said two ball clasps comprising a left ball clasp and a right ball clasp;
said left ball clasp comprising a first left ball clasp end and a second left ball clasp end;
said first left ball clasp end being securely embedded into said base;
said second left ball clasp end being located between first left premolar tooth and second left premolar tooth of said upper arch of teeth;
said right ball clasp comprising a first right ball clasp end and a second right ball clasp end;
said first right ball clasp end being securely embedded into said base;
said second right ball clasp end being located between first right premolar tooth and second right premolar tooth of said upper arch of teeth;
said two C-clasps comprising a left C-clasp and a right C-clasp;
said left C-clasp comprising a first left C-clasp end and a second left C-clasp end;
said first left C-clasp end being securely embedded into said base;
said second left C-clasp end clasping first left molar teeth of said upper arch of teeth;
said right C-clasp comprising a first right C-clasp end and a second right C-clasp end;
said first right C-clasp end being securely embedded into said base; and
said second right C-clasp end clasping first right molar teeth of said upper arch of teeth.

20. The removable and reusable oral detection device of claim 18, comprising
said tooth retainer in said mandibular style comprising a labial bow, two triangular clasps and two Adams claps;
said labial bow being a piece of metal wire;
said labial bow providing facial retention;
said labial bow comprising two ends securely embedded into said base;
said labial bow spanning from left canine tooth to right canine tooth of said lower arch of teeth;
said two triangular clasps comprising a left triangular clasp and a right triangular clasp;
said left triangular clasp comprising a first left triangular clasp end and a second left triangular clasp end;
said first left triangular clasp end being securely embedded into said base;
said second left triangular clasp end being located between first left premolar tooth and second left premolar tooth of said lower arch of teeth;
said right triangular clasp comprising a first right triangular clasp end and a second right triangular clasp end;
said first right triangular clasp end being securely embedded into said base;
said second right triangular clasp end being located between first right premolar tooth and second right premolar tooth of said lower arch of teeth;
said two Adams clasps comprising a left Adams clasp and a right Adams clasp;
said left Adams clasp comprising a first left Adams clasp end and a second left Adams clasp end;
said first left Adams clasp end being securely embedded into said base;
said second left Adams clasp end clasping first or second left molar tooth of said lower arch of teeth;
said right Adams clasp comprising a first right Adams clasp end and a second right Adams clasp end;
said first right Adams clasp end being securely embedded into said base; and
said second right Adams clasp end clasping first or second right molar teeth of said lower arch of teeth.
